(19) United States
(12) Patent Application Publication
(10) Pub. No.: US 2018/0182490 A1
(43) Pub. Date: Jun. 28, 2018

(54) Scoring and Mitigating Health Risks

(71) Applicant: BaseHealth, Inc., Redwood, CA (US)

(72) Inventors: Hossein Fakhrai-Rad, Los Altos, CA (US); Hadi Zarkoob, Sunnyvale, CA (US); Amir Bigdeli, San Francisco, CA (US); Shahin Gorgani, Redwood City, CA (US); Sarah Lewinsky, Fremont, CA (US); Harshna Kapashi, Mountain View, CA (US); Prakash Menon, Cupertino, CA (US); Suyash Rathi, Sunnyvale, CA (US)

(73) Assignee: BaseHealth, Inc., Redwood, CA (US)

(21) Appl. No.: 15/852,917

(22) Filed: Dec. 22, 2017

Related U.S. Application Data
(60) Provisional application No. 62/438,230, filed on Dec. 22, 2016.
(60) Provisional application No. 62/440,018, filed on Dec. 29, 2016.

(51) Int. Cl. G16H 50/30; G16H 50/50; G06Q 10/06

(52) U.S. Cl. CPC G16H 50/30; G06Q 10/06315; G16H 50/50

(57) ABSTRACT

Various areas of medicine and healthcare may benefit from improvements in the identification of and mitigation of health risks. For example, medicine and healthcare may benefit from systems and methods that can mine literature to select and analyze the risk factor(s) contributing to the development, progression and management of common health conditions. A method can include receiving an input health condition. The method can also include scoring the health condition based on a plurality of risk factor sources to generate a health score. The method can further include providing the health score and at least one remediation goal based on the risk factor sources.

### CROSS-REFERENCE TO RELATED APPLICATIONS

This application is a non-provisional of, and claims the benefit and priority of, U.S. Provisional Patent Application No. 62/440,018, filed Dec. 29, 2016. This application is also a non-provisional of, and claims the benefit and priority of, U.S. Provisional Patent Application No. 62/438,230, filed Dec. 22, 2016.

### BACKGROUND

### Field

Various areas of medicine and healthcare may benefit from improvements in the identification of and mitigation of health risks. For example, medicine and healthcare may benefit from systems and methods that can mine literature to select and analyze the risk factor(s) contributing to the development, progression and management of common health conditions.

### Description of the Related Art

In the healthcare industry, the presence of health risk factors is used to determine which individuals are at high risk of developing a health condition and should be recommended for intervention. It is important to determine both which risk factor(s) reliably affect the associated health condition risk and how much each risk factor contributes to the overall health condition progression.

Over the past decades, there have been many published studies that assess the risk factors for a multitude of health conditions. The challenge becomes how to sort through the available data and identify the risk factors that are statistically reliable and can be replicated in multiple populations and ethnicities. Searching through a database of references and abstracts about life sciences for such risk factors is a very time consuming process for even a single risk factor associated with a single health condition. This effort is compounded when comprehensively searching for all reliable risk factors for a large group of health conditions. The risk of selecting non-reliable risk factors as well as missing reliable risk factors for a health condition could be huge if this process is not handled in a structural and well-established format. It is essential to have a strict filtering methodology embedded in the pipeline which can efficiently and methodically reduce the large number of available scientific publications for each risk factor to a list of fine-tuned and reliable scientific publications per risk factor.

As new scientific data is published on a regular basis, it is also very important to keep up with all of the latest reliable scientific publications behind each risk factor for particular health conditions. New risk factor(s) or new scientific data for the existing risk factors can be identified as soon as this data is published. Obviously, there should be specific criteria that can weigh this new data set to determine whether the health condition assessment needs to be updated.

In order to reduce inefficiencies and increase the accuracy of manually mining and selecting scientific data from databases, utilizing automated learning systems including conventional natural language processing technologies is very valuable. Another improvement factor in this process is the utilization of medical experts' knowledge to confirm the final list of risk factors including the selected set of data for each risk factor.

The health analytics market has been a fast-growing area for many health-related companies (health plans, and accountable care organizations for example). However, the growth market has been mainly driven by health analytics tools focusing only on factors such as claims data and International Classification of Diseases (ICD) codes.

Furthermore, the claims and ICD codes data are primarily generated from individuals with at least one existing health condition and not the 93% of the population with health coverage who have not yet been diagnosed with a condition.

Moreover, the use of comprehensive health analytics tools in individuals and populations, provide significant growth opportunities in the market of total member prospective risk analysis, which is to stratify individuals and populations based on their risk level for various health conditions.

Lifestyle, medical, family history and genetic data in various ratios contribute towards the appearance and development of many known health conditions. There is not much one can do directly to control the genetic input for emergence of a health condition. However, by controlling the lifestyle factors one could postpone or even prevent the emergence of the condition.

In addition to personal information such as the age and gender of each individual, other types of data including lifestyle, medical, family history and genetic data might be available as part of individual's health profile. The lifestyle data may include diet, physical activity, and sleep among others. The medical data can be values of measurable parameters such as Body Mass Index (which, in one exemplary calculation, may be determined by BMI=weight in KG/Height in M2), blood pressure, and blood test data, among others. The family history data can be a report of any health condition that a first (parents and siblings) or second degree (grandparents, aunts and uncles) relative might have. The genetic data can be screening for Single Nucleotide Polymorphisms (SNPs) through genotyping, Exome Sequencing, whole genome sequencing, or any other technique to identify such genetic data.

To evaluate an individual's health status and score the level of risk the individual carries based on his/her health profile, healthcare professionals may use different standards, techniques or knowledge to provide that assessment. One healthcare professional might think a high systolic blood pressure (140 mmHg) is not an alarming risk factor for a 40-year-old male and the risk could be addressed by just cutting the salt consumption in his diet. However, another healthcare professional might believe the individual, based on his family history and other health profile data, needs also to be on antihypertensive medication in addition to reduced salt consumption in his diet. Furthermore, a systolic blood pressure of 140 mmHg might increase the risk of tens of different health conditions such as cardiovascular diseases, vascular diseases, kidney disorders, dementia/MCI, cancer and type 2 diabetes etc.; however, the impact of such a high level of systolic blood pressure might be different for each of these health conditions.

To evaluate and score the health status of a population, stratifying the population based on one single risk factor such as high BMI or in a combination of different ones such as high BMI and high systolic blood pressure might not necessarily capture all the individuals within the population who are potentially at the highest risk of developing serious conditions such as cardiovascular diseases, cancer and type 2 diabetes.

Some methods and systems for evaluating and interpreting health related data have been proven better than others over time and some experts have found better success than others. Additionally, new studies may provide a basis for improved methods for evaluating and interpreting health data and health management for many experts in many realms of health-related expertise.

### SUMMARY

According to certain embodiments, a method can include receiving an input health condition. The method can also include scoring the health condition based on a plurality of risk factor sources to generate a health score. The method can further include providing the health score and at least one remediation goal based on the risk factor sources.

In certain embodiments, an apparatus can include at least one processor and at least one memory including computer program code. The at least one memory and the computer program code can be configured to, with the at least one processor, cause the apparatus at least to receive an input health condition. The at least one memory and the computer program code can also be configured to, with the at least one processor, cause the apparatus at least to score the health condition based on a plurality of risk factor sources to generate a health score. The at least one memory and the computer program code can be further configured to, with the at least one processor, cause the apparatus at least to provide the health score and at least one remediation goal based on the risk factor sources.

A non-transitory computer-readable medium can be encoded with instructions that, when executed in hardware, perform a process. The process can include receiving an input health condition. The process can also include scoring the health condition based on a plurality of risk factor sources to generate a health score. The process can further include providing the health score and at least one remediation goal based on the risk factor sources.

### BRIEF DESCRIPTION OF THE DRAWINGS

For proper understanding of the invention, reference should be made to the accompanying drawings, wherein:

FIG. 1 illustrates the overall functionality of a DLM engine, according to certain embodiments of the present invention.

FIG. 2 illustrates an overall flow of selecting highly scored risk factors for a health condition, according to certain embodiments of the present invention.

FIG. 3 illustrates criteria for rejecting unacceptable scientific publications, according to certain embodiments of the present invention.

FIG. 4 illustrates elements that are utilized to score the quality of each publication, according to certain embodiments of the present invention.

FIG. 5 illustrates an exemplary overview of functionality of the Health Score System according to certain embodiments of the present invention including a list of data input and output.

FIG. 6 demonstrates five designed categories which are utilized to measure the Health Score, according to certain embodiments of the present invention.

FIG. 7 shows an exemplary flow of how the Health Score of certain embodiments of the present invention improves by time, including a list of contributing factors.

FIG. 8 illustrates four elements that are utilized for weighting an exemplary risk factor of certain embodiments of the present invention.

FIG. 9 illustrates exemplary statistical data modeling that has been used to measure the correlation between the % health risk and the actual risk factor value, according to certain embodiments of the present invention, including both categorical (step-wise) and continuous data modeling in this example.

FIG. 10 illustrates the tracking frequency for number of different actionable health risk factors which affect an exemplary Health Score value of certain embodiments of the present invention.

FIG. 11 illustrates an example of various elements in creating an example Health Score of certain embodiments of the present invention.

FIG. 12A illustrates a method according to certain embodiments of the present invention.

FIG. 12B illustrates a further method according to certain embodiments of the present invention.

FIG. 13 illustrates a system according to certain embodiments of the present invention.

### DETAILED DESCRIPTION

Unless otherwise defined, all terms (including technical and scientific terms) used herein have the same meanings as commonly understood by one having ordinary skill in the art to which this innovation belongs. It will be further understood that terms, such as those defined in commonly used dictionaries, should be interpreted as having a meaning that is consistent with their meaning in the context of the relevant art.

In the describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion.

A new health score system and methodology using a comprehensive set of health risk factors in an individual or in a population are discussed herein. In the following description, for purpose of explanation, numerous specific details are set forth in order to provide a thorough understanding of the present invention.

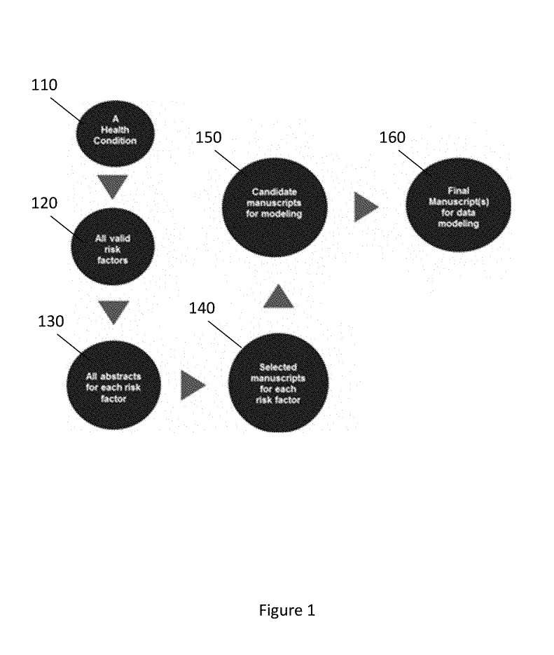

Certain embodiments of the present invention also include an intelligent and automated learning machine which can select the most applicable health publications for any risk factor related to any given health condition. Certain embodiments of the present invention are referred to as a data learning machine (DLM) as shown in FIG. 1. The DLM has been designed to overcome the challenges of data mining and data selection from both structured and non-structured sets of data.

The DLM at a high level can work as shown in FIG. 1. The name of a health condition of interest can be provided to the machine at 110. The DLM can then, at 120, search for all the associated risk factors for that condition utilizing several valid scientific data sources. Each of these data sources can carry a different score based on their scientific reliability. Also, depending on the number of times each risk factor is captured in these data sources, each risk factor can obtain a specific score. The larger the score is, the more reliable the selected risk factor can be considered to be.

Another input to the DLM can be a combination of a health condition's name and its associated high-scored risk factors to search for the relevant data. The machine can then, at 130, seek for all possible abstracts that demonstrate any level of association between the health condition and its associated risk factors.

Subsequently, in various implementations of the present invention, the machine can run the title and the content of each abstract through a black list and a number of criteria associated with high quality statistical data to eliminate non-specific abstracts and can save the ones with a high association score to yield selected manuscripts at 140. The manuscript for each of these high-quality abstracts can then, at 150, be selected by the DLM based on several other criteria such as sample size, type of the study, and statistical relevance among a number of additional selection criteria.

Furthermore, the DLM can select and download the top ranked and most reliable and relevant scientific manuscripts from the thousands initially selected abstracts for each risk factor as final manuscripts at 160. The machine can also generate a unique and extensive data file per risk factor with all the needed information from the selected manuscripts to model the risk factor utilizing an evidence-based risk predictor engine.

The DLM can facilitate the process of reviewing tens to millions of scientific publications within minutes and collecting the most scientifically reliable publications with the most appropriate and relevant information. The process described above can save effort from manually reading scientific publications, which can increase the level of data accuracy and consistency.

In addition to utilizing artificial intelligence, machine learning technologies, or similar statistical tools to learn relationships between numerous risk factors and health conditions, the DLM engine can also be trained with the knowledge from medical experts in this field who have the experience of doing this type of work for many years but in an old fashion mode. In fact, utilizing DLM is a dependable tool to get the most reliable scientific data, keep the science up-to-date and increase the efficiency and precision in selecting scientific data.

For the automated DLM to work accurately, artificial intelligence techniques can be mixed with field expertise. The engine input (for example, at 110 in FIG. 1) can be the names of health conditions and risk factors and the output can be high quality and applicable scientific publications (for example at 160 in FIG. 1). One important feature of an embodiment of a process of the present invention is an ontology list that can be generated to train the engine to detect the data of interest. The exemplary ontology list is the result of several years of compiling knowledge of medical terminology that is being used by the scientific community. In another way, the ontology utilized in aspects of the present invention will work as the backbone of the DLM for highly accurate data selection. Below, steps of the process are illustrated in FIG. 1.

There is some inconsistency in terminology of health data that is used across publications. Different publications may use different terms for both health conditions and risk factors. This could be a challenging problem when a researcher is trying to search for a specific risk factor for a health condition. There is always a possibility of missing an article if the engine cannot map its own health condition name to the health condition name being reported in the publication. In order to solve this problem or for other reasons, in various embodiments, a list of all the alternative names for risk factors and health conditions can be generated. In this list, the system can find and display multiple synonyms per risk factor or health condition. In various embodiments, a black list can also be generated. The DLM can utilize the black list to avoid adding any publications with any of the phrases on the black list. It is essential for various inventive features to train the engine in a way that only targets the types of publications that articulate the association between desired risk factors and health conditions.

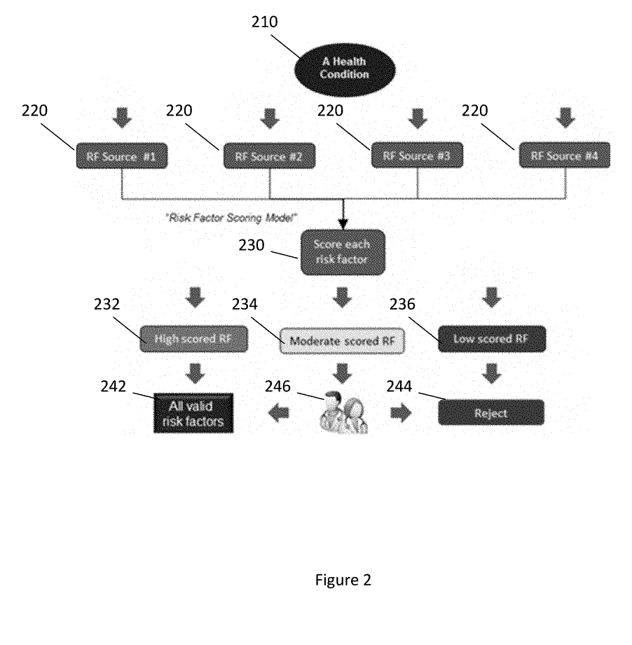

FIG. 2 illustrates a process for generating a risk factor list. The purpose of this step may be to generate a fully automated table of risk factors from different data sources based on the health condition of interest. In this step, an implementation of a script, for example, can use the illustrated steps to generate a risk factor list. For a given health condition 210, the DLM can utilize defined data sources 220, to generate a sorted list with all the most well established risk factors for the desired health condition.

The DLM can score each risk factor at 230, yielding high-scored risk factors 232, moderate-scored risk factors 234, and low-scored risk factors 236. The high-scored risk factors can be automatically identified as valid risk factors at 242, while low-scored risk factors can be automatically rejected at 244. Moderate-scored risk factors can be provided to an expert for manual review at 246.

Scoring risk factors can be done in a variety of ways. For example, risk factors reported in the most prominent data sources can get a higher score. Likewise, risk factors reported by multiple data sources can get higher scores. The DLM can record any citations associated with each risk factor from each source. Moreover, the DLM can sort the list based on the final score. The risk factors with the highest score can stand at the top of the list and can automatically be forwarded to the next step at 242, as mentioned above. Meanwhile, medical experts can, at 246, review the lower scored risk factors list before the list gets finalized.

The DLM can automatically search numerous scientific literature databases. In order to do so, search strings can be defined to target the right output. The engine can then search within the databases by utilizing an ontology list to capture publications that demonstrate the association between different health conditions and risk factors terminologies. At the end, the DLM compiles a list of outputs for the next step, which is publication evaluation.

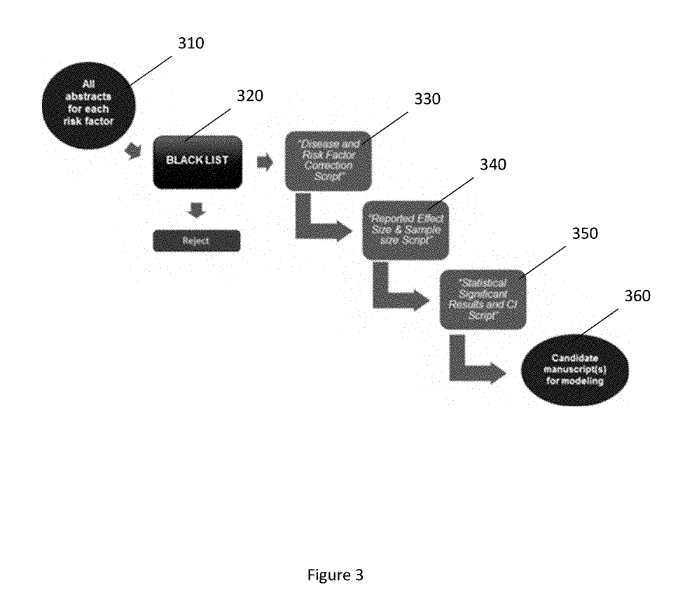

FIG. 3 illustrates publication evaluation. In publication evaluation, the DLM can analyze the resulting outputs from the data search. The goal in this phase may be to identify the right publications based on the title and the abstract of each article. The DLM can scan the title and abstract (received at 310) based on the criteria described below and can create a scoring list based on the quality of the publication. Also, certain publications may get rejected immediately if the title contains any of the phrases from the black list, at 320. This step can help to maximize the quality of the publications and their contents.

The DLM can scan the title and abstract with the following criteria to accept or reject: correct phenotype; correct risk factor at 330; approved statistical models at 350; large sample size at 350; non-statistically significance at 350; and no words from the black list at 320.

If the publications pass all the criteria above, then at 360 they will be downloaded in a Portable Document Format (PDF) and proceeds to the PDF scanning step.

Next, the DLM can scan the publications and gather the information defined in the list below. The purpose of this step may be to have the DLM bypass human intervention by scanning the file and locating the relevant data. The relevant information can include any of the following: sample size, study type, analysis type, diagnostic tool, exclusion criteria, gender, ethnicity, location, category name or range, category definition, risk value, significant confidence interval, and minimum/maximum/normal value of each risk factor.

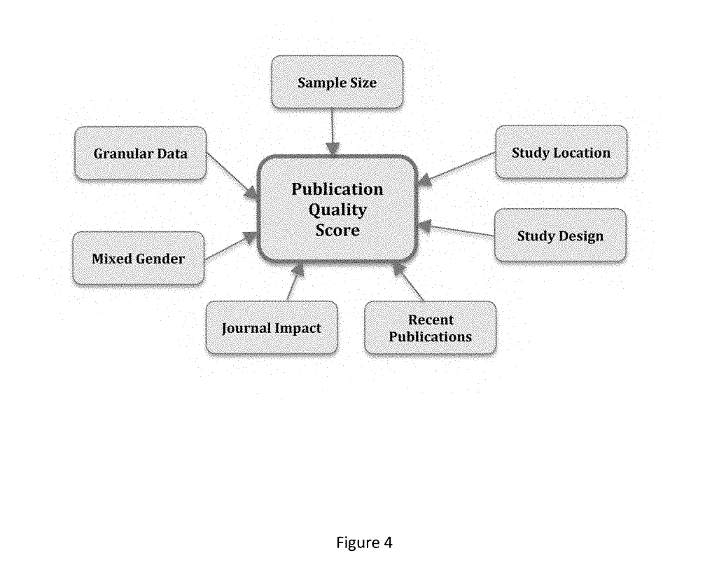

In the last step, illustrated in FIG. 4, one to several publications with the highest quality can be marked for the medical experts to select the best publications from. As shown in FIG. 4, some of the criteria used for selecting the final publications can include the following: sample size; granularity of data; type of populations in the study; prominence of the journal; date of the publication; diversity of gender; and whether the study is a prospective study.

In some cases, if there are multiple legitimate publications, their data can be combined and modeled through a META analysis approach. Papers with the highest score can get more weight in the META analysis.

A variety of health risk factors can be considered. A list of specific health risk factors under each risk categories (lifestyle, medical, family history, and genetics) may have been identified through an extensive scientific data mining and selection approach, such as the approach outlined above. The selected health risk factors under lifestyle and medical categories may be among the actionable factors which can be modified and/or improve over time. The selected health risk factors under family history and genetic categories on the other hand generally may not be modified over time. Nevertheless, these factors may significantly help the personalization of an assessment and generation of a health score.

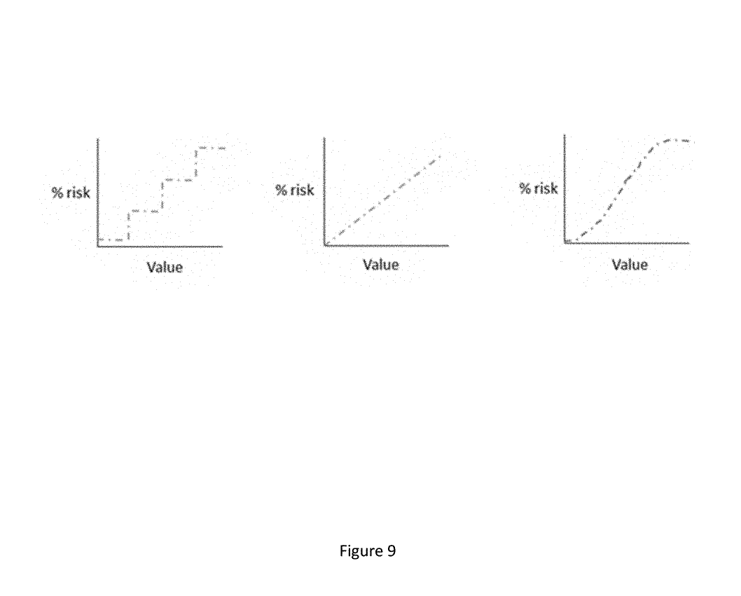

The size of the effect of each of these health risks can also be taken into account. In one aspect of certain embodiments of the present invention, the effect size in forms of Odds Ratio (OR), Hazard Ratio (HR) or Relative Risk Ratio (RR) for each health risk can be obtained through the approaches described above in regards to health risk factors and then modeled by applying categorical (step-wise) or continuous statistical modeling as shown in FIG. 9.

Certain embodiments of the present invention also include a model and system that can evaluate and score health risks in individuals and populations by using numerous health risk factors. These lifestyle, medical, family history and genetic risk factors can be utilized by the system in combinations as well as individually. The calculated health score for an individual or in a population may, in certain embodiments, be based on how many health conditions and health risk factors the individual is predisposed to. Each risk factor and health condition in the system has its own unique score from which the actual health score will be calculated by, for example, summing the score of each of these risk factors and health conditions. In one implementation, the health score is selected to be a number between 0 and 1000 in which the larger the scored number is, the healthier the individual is. The health score can then be stratified to specific actionable goals, which can be used and tracked by the individual to improve his/her health score. Depending on the weight and interaction level between each risk factor and each health condition, the frequency and extent of risk factor improvement will result in an enhancement in the health score.

In an embodiment of the present invention, total risk for a health condition can be calculated by utilizing the observed effect size for each health risk factor for that condition. Each condition has a unique set of associated health risk factors that can be taken from one or more risk category to complete the risk assessment.

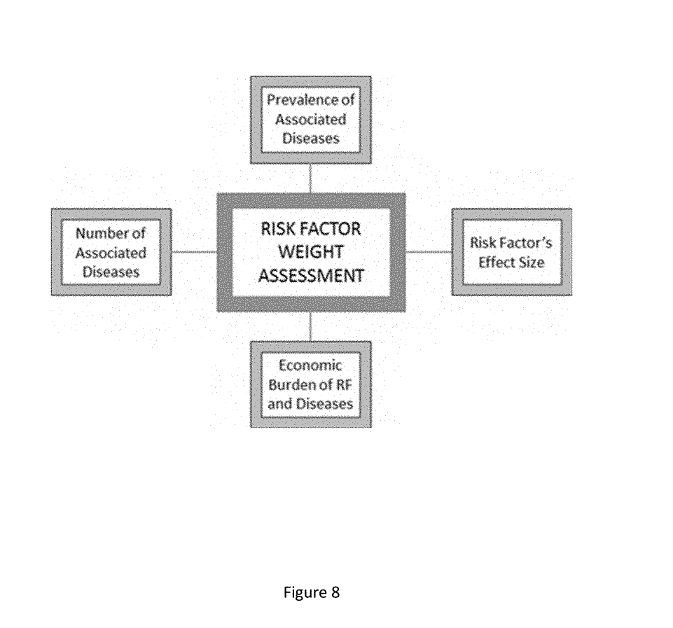

As the example of FIG. 8 shows, there can be four elements that contribute to the risk factor based assessment. A first element for each risk factor can be based on its association with the progression of a number of different health conditions. The more conditions the risk factor is associated with, the higher weight the risk factor may obtain. As a second element, the prevalence of associated conditions may also impact the weighting of each risk factor. The higher the condition prevalence is, the higher the risk factor weight may be. A third element that may increase the weight of a risk factor may be the high financial burden of having the risk factor and/or being predisposed for the health condition(s). A fourth element that may also increase the weight of a risk factor may be the actual effect size of the risk factor for the associated disease(s).

Other elements can also be utilized for health score calculation. In addition to the condition and risk factor based assessments discussed in the previous paragraphs, in certain embodiments three other elements can be involved in the final calculation of the individual's health score. The first one of these additional elements may be the completeness level of each health profile. A more complete health profile may contribute to a higher health score. A meaningful health score can be produced with either a complete set or a partially incomplete set of data. As more data is obtained for determination and tracking of the health score, its accuracy and statistical relevance may be improved. In this manner, an initial health score can be calculated, and its value can be corrected through the addition of complete sets of data. Certain initial estimates or population norms may be used to populate as-yet incomplete data. As more data is measured or obtained, the health score can be updated accordingly.

A second of these additional elements may be about the selection of actionable goal(s) that system has generated per individual. Selection of any actionable goal may also generate more health score per individual.

A third of these additional elements may be about the engagement with the selected actionable goal(s). A greater engagement with the selected actionable goal(s) through frequent tracking and monitoring effort may also contribute to a higher health score per individual.

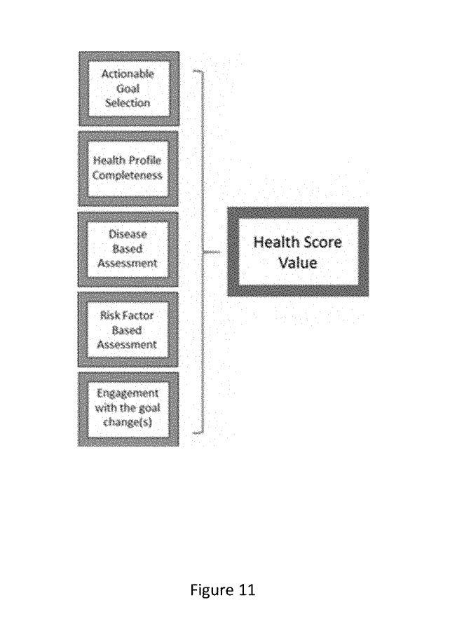

In certain embodiments, the condition-based assessments and risk factor assessments are two elements that contribute the most to the creation of the health score. The risk factor value contribution may be slightly lower compared to the condition based value. In certain of the embodiments illustrated by way of example, the health profile completeness may contribute a smaller range of the total health score, as may the engagement with the goal changes element. Actionable goal selection(s) is shown with the lowest contribution to the total health score. Examples of these embodiments may be seen in FIG. 11, and other configurations of factors contributing to the health score may be envisioned, including fewer or more factors, and different contributions/weights to the final health score. The contribution factor for these 5 elements described above might, for example, vary from 3% to 50%.

The calculation of a population health score can be based on an accumulation of all the individual health scores within that population. In various aspects of certain embodiments of the present invention, a series of subpopulations with different ranges of health scores along with a set of unique and actionable goals can be generated. The subpopulation's health scores can be weighted inter & intra between various risk factors and may be not scored based on selecting all the individuals with the highest risk value for an individual risk factor. The population health scores may allow, for example, health plans to identify the most risky and unhealthy subpopulation(s) and then target them with the most personalized interventions.

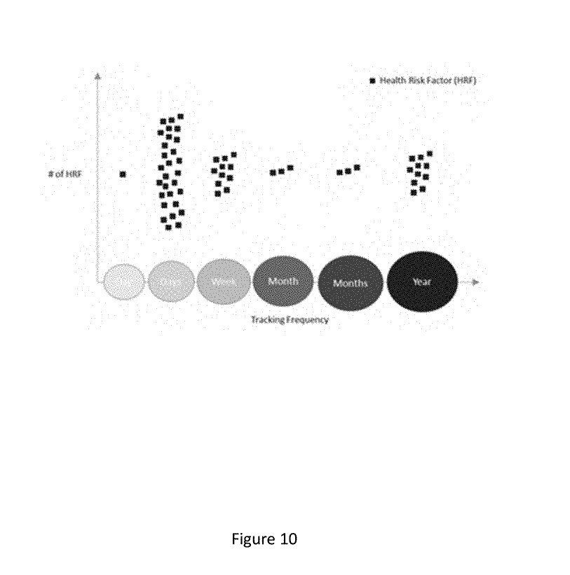

The risk factor-based assessment can generate a list of health risk factor(s) for each individual that can be monitored, tracked, and improved over time. The tracking of each health risk factor can have its own time sensitive schedule. It can start from a daily tracking schedule for some of these factors all the way to yearly basis tracking schedule for some other factors (see FIG. 10). This schedule may also depend on whether an individual needs to improve the risk factor or not.

Even if an individual has a healthy risk factor value it, may still be expected that they track, although at a lower frequency than those with unhealthy values, to ensure that their health profile data remains updated and accurate. For example, for a health risk factor with a weekly tracking schedule, the health score may not get further improved if an individual tracks the factor more than once per week. On the other hand, if a health risk factor that is supposed to be tracked weekly does not get tracked each week, the health score might be adversely affected.

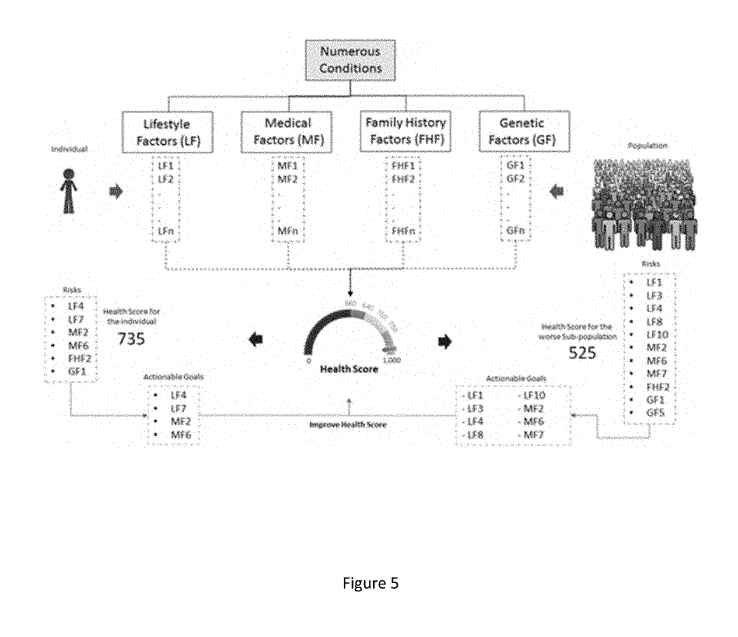

Various embodiments of the present invention may utilize the following steps. For example, as shown in FIG. 5, a risk factor based assessment can generate a list of health risk factors that need to be improved per individual. Numerous conditions can be considered. These factors may include lifestyle factors, medical factors, family history factors, and genetic factors. This can be performed either as to an individual (as shown at left) or as to a population. The output of the evaluation can be a numeric health score and a set of actionable goals.

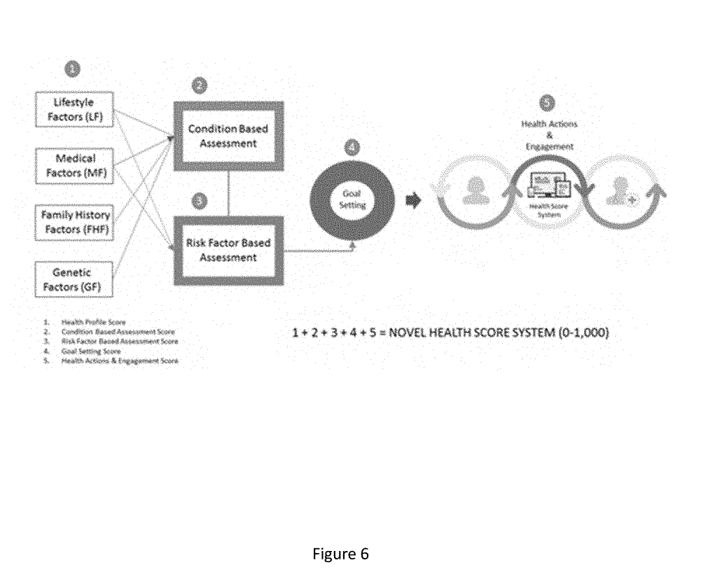

FIG. 6 similarly shows a health score system in more detail. As shown in FIG. 6, at 1 there can be various risk factors considered, including lifestyle factors, medical factors, family history factors, and genetic factors. All of these factors can be taken into account at 2 in a condition-based assessment. At 3, a risk factor based assessment can take into account the result of the condition-based assessment. The risk factor based assessment can also directly take into account lifestyle factors and medical factors.

At 4, there can be goal setting that takes the risk factor based assessment as its input. Then, at 5, there can be health actions and engagement. As described above, these can involve periodically reevaluating a health score using the health score system described above.

There are options for selecting any number of health risk factors to track and monitor. An individual can choose to track and monitor most risk factors, even those that the individual does not need to improve. Even healthy individuals may track (for example, on a less frequent schedule) to ensure that their health profile information stays updated and accurate.

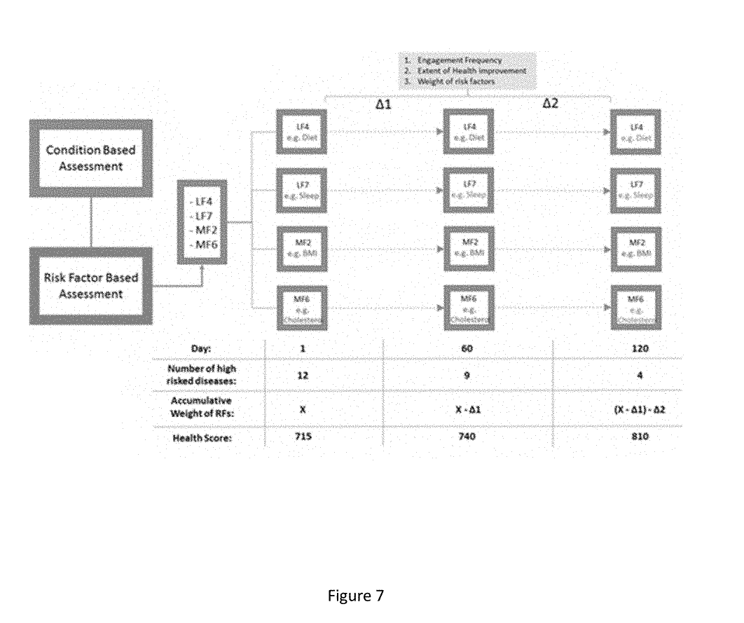

FIG. 7 illustrates how tracking may occur over time. As shown at the left, a condition based assessment and risk factor based assessment may yield a number of risk factors for tracking. These may be monitored as to engagement frequency for actions to mitigate the risk, extent of health improvement, and weight of the various risk factors. The result may show an improvement over time, for example if the individual takes corrective actions to reduce risk of disease.

As shown in FIG. 8, a risk factor weight assessment can take into account various factors. For example, the assessment can take into account number of associated diseases, prevalence of those associated diseases, a risk factor's effect size, and the economic burden associated with the risk factor and diseases. Non-economic considerations can also be made, but these may be hard to compare or balance with the economic considerations. The economic considerations may be particularly valuable in evaluating populations, for policy considerations either by governments or insurance companies.

As shown in FIG. 9 there can be various ways of linking a risk percentage to a value. FIG. 9 shows (from left to right), a stepwise function, a linear function, and a non-linear or asymptotic function. Any of these are possible. Moreover, multiple models can be combined. For example, a value of 0 may be used if a risk percentage is below some threshold, and then a value may increase linearly after that threshold.

The selected health risk factors can be tracked and monitored per each factor's tracking schedule. Depending on how unhealthy the value for each health risk factor is, each factor can still get improved when the provided personalized goal(s) for each risk factor has been achieved. Any positive improvement in the value of the given risk factor would positively impact the risk factor based assessment and potentially the condition based assessment which are the two most important contributing factors to an improved health score value.

FIG. 10 illustrates how different risk factors may be associated with different tracking frequencies. In this example, one risk factor is tracked daily (perhaps blood pressure or blood sugar, for example), while many risk factors may be tracked every few days, some risk factors may be tracked once a week, a few may be tracked once a month or once every few months. Finally, some risk factors may be tracked yearly or less frequently.

If the cumulative weight of a group of risk factors for an individual is initially x, the weight of the same group of risk factors in ti can be measured by subtracting Δ₁ which is the improved level of risk factors value from x. See FIG. 7 for illustration of an example.

Depending on what risk factor(s) has been targeted and what level of risk value improvement has been obtained, the risk for numerous conditions can be reduced at the same time. This may significantly improve the initially obtained health score.

As shown in FIG. 11, the health score value may be based on an actionable goal selection, health profile completeness, a disease-based assessment, a risk-factor based assessment, and engagement with any goal change(s) that have previously been assigned.

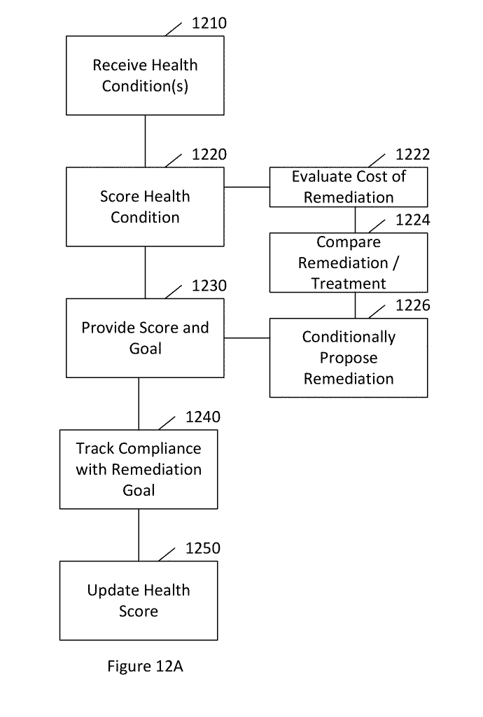

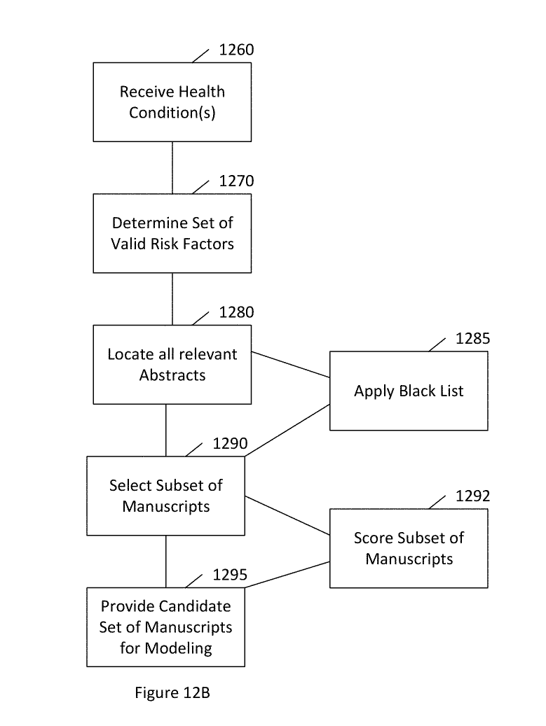

FIG. 12A illustrates a method according to certain embodiments of the present invention. As shown in FIG. 12A, a method can include, at 1210, receiving an input health condition. The input health condition can be an individual health condition or the health condition of a population. See, for example, FIGS. 2 and 7. More than one health condition can be considered. For example, multiple identified diseases, symptoms, or other factors can be considered at the same time.

As shown in FIG. 12A, the method can also include, at 1220, scoring the health condition based on a plurality of risk factor sources to generate a health score. The plurality of risk factor sources can include health risks based on lifestyle, medical, family history and genetic data. The scoring can include scoring each of the plurality of risk factor sources individually and combining the scores to provide an aggregate score. The scoring can include computing and utilizing one or more a health profile score, a condition-based assessment score, a risk factor based assessment score, a goal setting score, and a health actions and engagement score, as described above.

The method can include, at 1222, evaluating, for a population, a cost of implementing a remediation goal. This may, for example, be a cost associated with performing a preventative treatment such as a vaccine, or a wellness program such paying for a gym membership. In a certain case, an individual may perform this same evaluation. In this case, the population can be considered to be a population of one person, while in other cases the population may be a group of people, such as a family, the workers of an employer, the members of a health plan, or the residents of a governed area, such as a country.

The method can also include, at 1224, comparing the cost of implementing the remediation goal with an economic cost of treating the health condition without implementing remediation goal. Moreover, the method can further include, at 1226, proposing the remediation goal when the cost of implementing the remediation goal is lower.

The method can further include, at 1230, providing the health score and at least one remediation goal based on the risk factor sources. The method can additionally include, at 1240, tracking compliance with the remediation goal. The method can also include, at 1250, updating the health score based on a level or degree of compliance with the remediation goal.

FIG. 12B illustrates a further method according to certain embodiments of the present invention. FIG. 12B may be implemented using the approach shown and described with reference to FIGS. 1-4. As shown in FIG. 12B, a method can include, at 1260, receiving an input health condition. The method can also include, at 1270, determining a set of valid risk factors for the health conditions. The method can further include, at 1280, locating all abstracts in a database corresponding to each of the valid risk factors. The method can additionally include, at 1290, selecting a subset of manuscripts based on the abstracts. The method can also include, at 1295, providing a candidate set of manuscripts from the subset of manuscripts for modeling.

The method can also include, at 1285, applying a black list to the located abstracts to avoid including corresponding manuscripts in the selected subset of manuscripts.

The method can further include, at 1292, scoring the subset of manuscripts based on a plurality of factors, wherein a highest-scoring portion of the scored manuscripts are provided as the candidate set for modeling. The plurality of factors can include granularity of data, sample size, study location, study design, date of publication, impact of journal, diversity of gender, or any combination thereof.

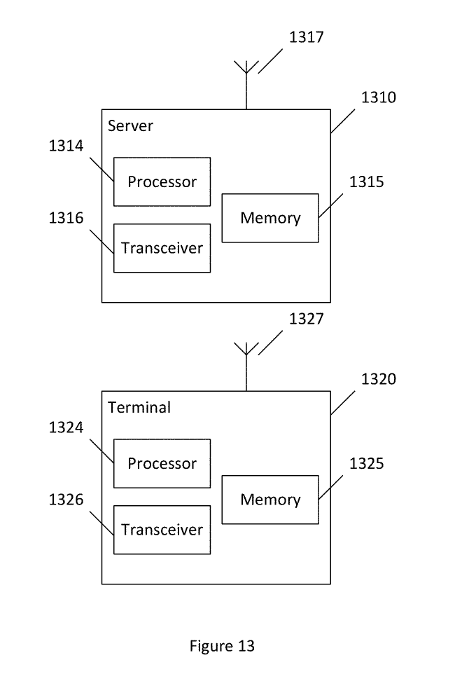

FIG. 13 illustrates a system according to certain embodiments of the invention. It should be understood that each block of the flowchart of FIG. 12A or 12B may be implemented by various means or their combinations, such as hardware, software, firmware, one or more processors and/or circuitry. In one embodiment, a system may include several devices, such as, for example, server 1310 and terminal 1320. The system may include more than one terminal 1320 and more than one server 1310, although only one of each is shown for the purposes of illustration. A server can be an application server, database, or combination thereof.

Each of these devices may include at least one processor or control unit or module, respectively indicated as 1314 and 1324. At least one memory may be provided in each device, and indicated as 1315 and 1325, respectively. The memory may include computer program instructions or computer code contained therein, for example for carrying out the embodiments described above. One or more transceiver 1316 and 1326 may be provided, and each device may also include an antenna, respectively illustrated as 1317 and 1327. Other configurations of these devices, for example, may be provided. For example, server 1310 and terminal 1320 may be solely configured for wired communication, and in such a case antennas 1317 and 1327 may illustrate any form of communication hardware, without being an antenna.

Transceivers 1316 and 1326 may each, independently, be a transmitter, a receiver, or both a transmitter and a receiver, or a unit or device that may be configured both for transmission and reception.

A terminal 1320 may be a mobile phone or smart phone or multimedia device, a computer, such as a tablet, or a personal data or digital assistant (PDA). In an exemplifying embodiment, an apparatus, such as a server or terminal, may include means for carrying out embodiments described above in relation to FIG. 12A or 12B.

Processors 1314 and 1324 may be embodied by any computational or data processing device, such as a central processing unit (CPU), digital signal processor (DSP), application specific integrated circuit (ASIC), programmable logic devices (PLDs), field programmable gate arrays (FPGAs), digitally enhanced circuits, or comparable device or a combination thereof. The processors may be implemented as a single controller, or a plurality of controllers or processors. Additionally, the processors may be implemented as a pool of processors in a local configuration, in a cloud configuration, or in a combination thereof. The term circuitry may refer to one or more electric or electronic circuits. The term processor may refer to circuitry, such as logic circuitry, that responds to and processes instructions that drive a computer.

For firmware or software, the implementation may include modules or units of at least one chip set (e.g., procedures, functions, and so on). Memories 1315 and 1325 may independently be any suitable storage device, such as a non-transitory computer-readable medium. A hard disk drive (HDD), random access memory (RAM), flash memory, or other suitable memory may be used. The memories may be combined on a single integrated circuit as the processor, or may be separate therefrom. Furthermore, the computer program instructions may be stored in the memory and which may be processed by the processors can be any suitable form of computer program code, for example, a compiled or interpreted computer program written in any suitable programming language. The memory or data storage entity is typically internal but may also be external or a combination thereof, such as in the case when additional memory capacity is obtained from a service provider. The memory may be fixed or removable.

The memory and the computer program instructions may be configured, with the processor for the particular device, to cause a hardware apparatus such as server 1310 and/or terminal 1320, to perform any of the processes described above (see, for example, FIG. 12A or 12B). Therefore, in certain embodiments, a non-transitory computer-readable medium may be encoded with computer instructions or one or more computer program (such as added or updated software routine, applet or macro) that, when executed in hardware, may perform a process such as one of the processes described herein. Computer programs may be coded by a programming language, which may be a high-level programming language, such as objective-C, C, C++, C#, Java, etc., or a low-level programming language, such as a machine language, or assembler. Alternatively, certain embodiments of the invention may be performed entirely in hardware.

Furthermore, although FIG. 13 illustrates a system including a server 1310 and a terminal 1320, embodiments of the invention may be applicable to other configurations, and configurations involving additional elements, as illustrated and discussed herein. For example, multiple terminals and multiple servers may be present, or other nodes providing similar functionality.

One having ordinary skill in the art will readily understand that the invention as discussed above may be practiced with steps in a different order, and/or with hardware elements in configurations which are different than those which are disclosed. Therefore, although the invention has been described based upon these preferred embodiments, it would be apparent to those of skill in the art that certain modifications, variations, and alternative constructions would be apparent, while remaining within the spirit and scope of the invention.

We claim:

1. A method, comprising:
receiving an input health condition;
scoring the health condition based on a plurality of risk factor sources to generate a health score; and
providing the health score and at least one remediation goal based on the risk factor sources.

2. The method of claim 1, wherein the input health condition comprises an individual health condition.

3. The method of claim 1, wherein the input health condition comprises health condition of a population.

4. The method of claim 1, wherein the plurality of risk factor sources comprise health risks based on lifestyle, medical, family history and genetic data.

5. The method of claim 1, wherein the scoring comprises scoring each of the plurality of risk factor sources individually and combining the scores to provide an aggregate score.

6. The method of claim 1, wherein the scoring comprises computing and utilizing one or more a health profile score, a condition-based assessment score, a risk factor based assessment score, a goal setting score, and a health actions and engagement score.

7. The method of claim 1, further comprising:
tracking compliance with the remediation goal; and
updating the health score based on a level or degree of compliance with the remediation goal.

8. The method of claim 1, further comprising:
evaluating, for a population, a cost of implementing the remediation goal;
comparing the cost of implementing the remediation goal with an economic cost of treating the health condition without implementing remediation goal; and
proposing the remediation goal when the cost of implementing the remediation goal is lower.

9. An apparatus, comprising:
at least one processor; and
at least one memory including computer program code,
wherein the at least one memory and the computer program code are configured to, with the at least one processor, cause the apparatus at least to
receive an input health condition;
score the health condition based on a plurality of risk factor sources to generate a health score; and
provide the health score and at least one remediation goal based on the risk factor sources.

10. The apparatus of claim 9, wherein the input health condition comprises an individual health condition.

11. The apparatus of claim 9, wherein the input health condition comprises health condition of a population.

12. The apparatus of claim 9, wherein the plurality of risk factor sources comprise health risks based on lifestyle, medical, family history and genetic data.

13. The apparatus of claim 9, wherein the scoring comprises scoring each of the plurality of risk factor sources individually and combining the scores to provide an aggregate score.

14. The apparatus of claim 9, wherein the scoring comprises computing and utilizing one or more a health profile score, a condition-based assessment score, a risk factor based assessment score, a goal setting score, and a health actions and engagement score.

15. The apparatus of claim 9, wherein the at least one memory and the computer program code are configured to, with the at least one processor, cause the apparatus at least to
track compliance with the remediation goal; and
update the health score based on a level or degree of compliance with the remediation goal.

16. The apparatus of claim 9, wherein the at least one memory and the computer program code are configured to, with the at least one processor, cause the apparatus at least to
evaluate, for a population, a cost of implementing the remediation goal;
compare the cost of implementing the remediation goal with an economic cost of treating the health condition without implementing remediation goal; and
propose the remediation goal when the cost of implementing the remediation goal is lower.

17. A non-transitory computer-readable medium encoded with instructions that, when executed in hardware, perform a process, the process comprising:
receiving an input health condition;
scoring the health condition based on a plurality of risk factor sources to generate a health score; and
providing the health score and at least one remediation goal based on the risk factor sources.

18. The non-transitory computer-readable medium of claim 17, the process further comprising:
tracking compliance with the remediation goal; and
updating the health score based on a level or degree of compliance with the remediation goal.

19. The non-transitory computer-readable medium of claim 17, the process further comprising:
evaluating, for a population, a cost of implementing the remediation goal;
comparing the cost of implementing the remediation goal with an economic cost of treating the health condition without implementing remediation goal; and
proposing the remediation goal when the cost of implementing the remediation goal is lower.

20. A method, comprising:
receiving an input health condition;
determining a set of valid risk factors for the health conditions;
locating all abstracts in a database corresponding to each of the valid risk factors;
selecting a subset of manuscripts based on the abstracts; and
providing a candidate set of manuscripts from the subset of manuscripts for modeling.

21. The method of claim 20, further comprising:
applying a black list to the located abstracts to avoid including corresponding manuscripts in the selected subset of manuscripts.

22. The method of claim 20, further comprising:
scoring the subset of manuscripts based on a plurality of factors, wherein a highest-scoring portion of the scored manuscripts are provided as the candidate set for modeling.

23. The method of claim 22, wherein the plurality of factors comprise granularity of data, sample size, study location, study design, date of publication, impact of journal, and diversity of gender.

24. An apparatus, comprising:
at least one processor; and
at least one memory including computer program code,
wherein the at least one memory and the computer program code are configured to, with the at least one processor, cause the apparatus at least to
receive an input health condition;
determine a set of valid risk factors for the health conditions;
locate all abstracts in a database corresponding to each of the valid risk factors;
select a subset of manuscripts based on the abstracts; and
provide a candidate set of manuscripts from the subset of manuscripts for modeling.

25. The apparatus of claim 24, further comprising:
applying a black list to the located abstracts to avoid including corresponding manuscripts in the selected subset of manuscripts.

26. The apparatus of claim 24, further comprising:
scoring the subset of manuscripts based on a plurality of factors, wherein a highest-scoring portion of the scored manuscripts are provided as the candidate set for modeling.

27. The apparatus of claim 26, wherein the plurality of factors comprise granularity of data, sample size, study location, study design, date of publication, impact of journal, and diversity of gender.

28. A non-transitory computer-readable medium encoded with instructions that, when executed in hardware, perform a process, the process comprising:
receiving an input health condition;
determining a set of valid risk factors for the health conditions;
locating all abstracts in a database corresponding to each of the valid risk factors;
selecting a subset of manuscripts based on the abstracts; and
providing a candidate set of manuscripts from the subset of manuscripts for modeling.

29. The non-transitory computer-readable medium of claim 28, further comprising:
applying a black list to the located abstracts to avoid including corresponding manuscripts in the selected subset of manuscripts.

30. The non-transitory computer-readable medium of claim 28, further comprising:
scoring the subset of manuscripts based on a plurality of factors, wherein a highest-scoring portion of the scored manuscripts are provided as the candidate set for modeling.

31. The non-transitory computer-readable medium of claim 30, wherein the plurality of factors comprise granularity of data, sample size, study location, study design, date of publication, impact of journal, and diversity of gender.
